(19) United States
(12) Patent Application Publication
(10) Pub. No.: US 2016/0174923 A1
(43) Pub. Date: Jun. 23, 2016

(54) NEUTRON GENERATOR FOR MEASURING TRACE ELEMENTS IN TISSUE

(71) Applicant: Purdue Research Foundation, West Lafayette, IN (US)

(72) Inventors: Linda Nie, West Lafayette, IN (US); Yingzi Liu, West Lafayette, IN (US)

(73) Assignee: Purdue Research Foundation, West Lafayette, IN (US)

(21) Appl. No.: 14/977,231

(22) Filed: Dec. 21, 2015

Related U.S. Application Data
(60) Provisional application No. 62/094,291, filed on Dec. 19, 2014.

(51) Int. Cl. A61B 6/00; G01T 1/24; A61B 6/10

(52) U.S. Cl. CPC A61B 6/505; A61B 6/107; A61B 6/40; A61B 6/4258; A61B 6/5205; G01T 1/24

(57) ABSTRACT

Disclosed herein is an in vivo neutron activation analysis (NAA) system comprising a neutron generator, a moderator operably coupled to the neutron generator, a cavity, a reflector operably coupled to the neutron generator, moderator, and cavity, and a shielding operably coupled to the neutron generator, moderator, cavity, and reflector. The system provides in vivo analysis of one or more trace elements stored in tissues after exposure.

### CROSS-REFERENCE TO RELATED APPLICATIONS

The present patent application is related to and claims the priority benefit of U.S. Provisional Patent Application No. 62/094291, filed Dec. 19, 2014, the contents of which is hereby incorporated by reference in its entirety into this disclosure.

This invention was made with government support under OH010044 awarded by the

Center for Disease Control and Prevention. The government has certain rights in the invention.

### TECHNICAL FIELD

The present disclosure generally relates measuring trace elements in tissue, and in particular to a device and method for measuring trace elements in human or animal tissue using characteristic gamma rays.

### BACKGROUND

This section introduces aspects that may help facilitate a better understanding of the disclosure. Accordingly, these statements are to be read in this light and are not to be understood as admissions about what is or is not prior art.

Over the past several decades there have been many reports linking exposure and inclusion of trace elements into tissues with diseases. Currently, there are no portable devices available for measuring some of these trace elements in live subject's tissue.

For example, manganese (Mn) is an essential trace element in the human body. Adverse health effects occur when body Mn storage is either too low or too high. Mn deficiency is generally not recognized among humans because of their diverse diets. Yet, Mn overexposure is common. Occupational exposure to Mn often takes place in mining, welding, steel industry, and other industrial settings. Environmental exposure to Mn has been reported in the uses of Mn-containing products (e.g. Mn-based pesticides), contamination in drinking water and food, and the use of Mn compounds in gasoline. There are also reports of excessive Mn exposure among ephedron drug abusers.

With excessive Mn deposition, chronic Mn toxicity can be evidenced in cardiovascular, liver, reproductive, and developmental problems, although it is mainly seen to affect lung tissue and the central nervous system. Indeed, workers exposed to Mn have reported various neurological disorders, including poor eye-hand coordination, reduced cognitive flexibility, tremors, and poor postural stability. In severe cases, a devastating neurological impairment called “manganism” occurs. At lower levels, Mn exposure causes more subtle neurological disorders. The symptoms of chronic Mn toxicity usually become progressive and are irreversible, reflecting permanent damage to neurological structure.

Mn levels in the human body have been estimated from blood, serum, urine, hair, and toenails. There is evidence that these biomarkers are useful in some ways. For instance, blood, hair, and toenail Mn concentrations were found to be higher in occupationally exposed workers than in matched controls in different studies. However, these biomarkers are of little use for long-term cumulative exposure assessment. Analyses of whole blood Mn were found to be highly variable among human population and are not significantly correlated with Mn-induced neurotoxicity. This is due primarily to a short half-life of Mn in blood and large intracellular distribution. High variability was also observed for Mn concentrations in toenails and hair. In addition hair and nail samples are subject to external contamination. Recently, MRI imaging technologies have been developed to quantify Mn in brain tissue using the signal intensity in the basal ganglia region. While it is advantageous to directly relate Mn exposure to brain function, this technology again has the limitation that Mn is released from the brain after a short time, and hence it only reflects recent Mn exposure. Overall, the lack of a reliable cumulative exposure biomarker limits the capacity for epidemiologic studies to detect a relationship between Mn exposure and neurodegeneration, especially for low-level exposures and their more subtle neurological effects.

On the other hand, bone has much greater potential to be a reliable biomarker for cumulative Mn in the body. Schroeder et al. reported an average Mn concentration of 2 μg/g in bone ash, which gives rise to about 32.5% of body Mn being contained in bone, according to our previous calculation. International Commission on Radiological Protection (ICRP) reported about 40% of body Mn in bone. A recent study shows that the average half-life of Mn in bone is 147 days in rats that are chronically exposed to Mn-contaminated drinking water, which equivalent to the half-life of Mn in human bone of about 8 years. The same researchers also show a significant correlation between brain Mn and bone Mn in rats fed with Mn-contained water.

Hence, there is a need for a device and method to accurately measure the trace elements, like manganese, in vivo.

### SUMMARY

According to one aspect, an in vivo neutron activation analysis (NAA) system is presented, comprising, a neutron generator, a moderator operably coupled to the neutron generator, a cavity, a reflector operably coupled to the neutron generator, moderator, and cavity, a shielding operably coupled to the neutron generator, moderator, cavity, and reflector. The neutron generator may be positioned vertically. The shielding includes components may comprise high density polyethylene (HDPE), borated HDPE, and lead. The shielding may be configured to lock or snap together. The neutron generator may a range of neutrons between about 1×10⁸ to 1×10¹¹ neutrons per second. The moderator may include components made of polyethylene (PE). The reflector may include components made of graphite or polyethylene (PE).

### BRIEF DESCRIPTION OF THE DRAWINGS

FIG. 1 is an image of a deuterium-deuterium (DD) neutron generator.

FIG. 2 is a schematic plot of the DD neutron generator head.

FIG. 3 is a cross section of the in vivo neutron activation system (INAS) system according to one embodiment.

FIG. 4 shows a perspective view of the neutron activation analysis system of FIG. 3.

FIG. 5A shows a perspective view of the neutron activation analysis system according to one embodiment.

FIG. 5B shows a perspective view of the neutron activation analysis system during in vivo measurement.

FIG. 5C shows a second perspective view of the neutron activation analysis system.

FIG. 6 is an image of a Detector Unit with high-efficiency HPGe detectors.

FIG. 7 shows a schematic of the reaction when a neutron excites Manganese and the resulting products.

FIG. 8 is a graph showing Mn gamma-ray spectrum from on the 100% efficiency HPGe detectors for the hand phantom doped with 5 ppm Mn.

FIG. 9 is a graph showing a preliminary calibration line with 5 concentrations.

FIG. 10 is a graph showing neutron fluence spectrum for different moderators.

FIG. 11 is a graph showing neutron fluence spectrum for different reflectors.

FIG. 12 is a Gamma-ray spectrum of a bone-equivalent phantom doped with 5 ppm Mn.

FIG. 13 is a graph showing Mn/Ca versus Mn concentration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The disclosure provides details on a new system and method for in vivo analysis of one or more trace elements stored in tissues after exposure. In some embodiments the tissue is soft tissue. In other embodiments the tissue is bone or cartilage. According to one embodiment, a system comprises an irradiation device or in vivo neutron activation system (INAS) unit 100 and an analysis device or Detector Unit 101.

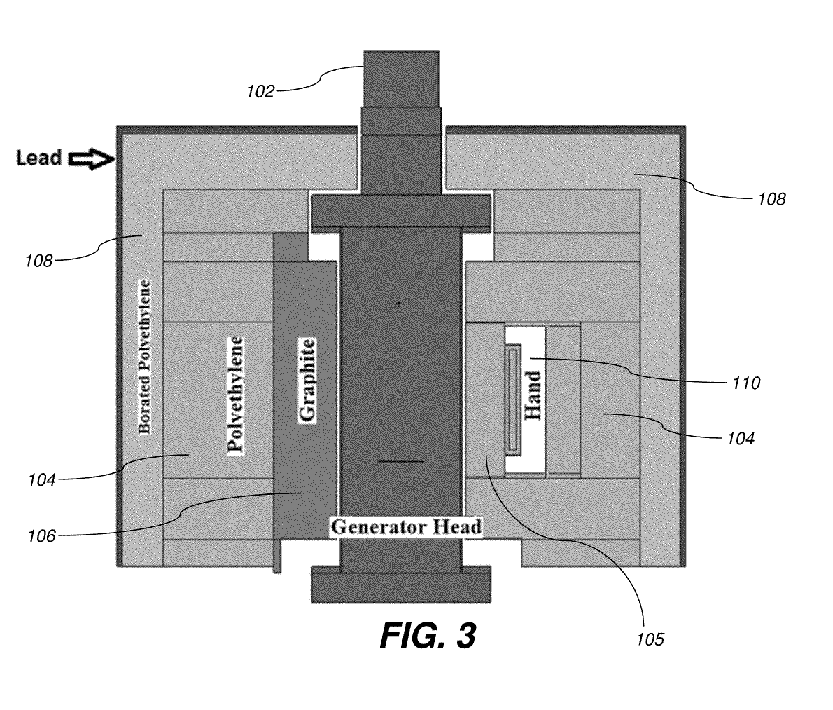

The INAS unit comprises a neutron generator 102, a moderator 105, reflectors 104 and 106, a shielding 108, and a cavity 110 as shown in the simulation model of FIG. 3. The neutron generator 102 generates a range of neutrons between about 1×10⁸ to 1×10¹¹ neutrons per second. In some embodiments the neutron generator 102 may generate between about 1×10⁶ to about 1×10¹³ neutrons per second. In certain embodiments the neutron generator 102 is a compact deuterium-deuterium (DD) neutron generator.

The neutron generator 102 is operably coupled to the moderator105 and reflectors 104 or 106. The shielding 108 surrounds the neutron generator 102, reflectors 104 and 106, moderator 105 and cavity 110. In certain embodiments there may be a cooling element to cool the neutron generator while it's operating.

The reflectors 104 and 106 may be made in any desired shape including but not limited to a rectangle, square, oval, circle, triangle, or any shape curved around the neutron generator, and from a variety of materials including, polyethylene, paraffin, graphite, or a combination thereof.

The moderator 105 may be made in any desired shape including but not limited to a rectangle, square, oval, or circle and from a variety of materials including, polyethylene, paraffin, or a combination thereof.

The shielding 108 may comprise a high density polyethylene (HDPE), borated HDPE, lead, or a combination thereof. The shielding 108 may be one solid piece or it may be a plurality of pieces. The pieces may fit together in a LEGO-like fashion (e.g., interlocking blocks) to remove any gaps and provide structural stability or they may be freely placed pieces. The shielding may be a single piece or a plurality of pieces which are then covered by an outer casing.

The Detector Unit 101 comprises at least one detector, shielding, and a computer. The Detector Unit 101 may comprise up to six detectors to increase sensitivity.

The tissue may come from any source including, any mammal, invertebrate, or tissue culture. The tissue source may be from a living source or non-living source.

By “vertebrates” refers to any animal species that possesses or develops a vertebral column including but not limited to mammals, humans, mice, dogs, cows, horses, cats, fish, whales, sharks etc.

By “invertebrate” refers to are animal species that neither possess nor develop a vertebral column, and includes insects, crabs, lobsters, worms, etc.

By “tissue culture” refers to tissue or cells grown separately from an organism in a laboratory setting. For example, a synthetic or tissue-culture grown organ or tissue may be of interest to test as a model for exposure to certain trace elements in an environment.

By “trace elements” refers to aluminum, manganese, magnesium, iron, titanium, fluorine, gadolinium, calcium, chromium, nickel, copper, zinc, cadmium, indium, gold, mercury, silver, chlorine, potassium, sodium, phosphorous, sulfur, or other trace elements.

By “subject” refers to the tissue and source being dosed and/or analyzed.

The INAS 100 and the Detector Unit 101 may be separate devices or located in the same device. In certain embodiments the cavity in the INAS 100 may be modified or inherently includes the components of the Detector System 101 for irradiating and analyzing in the same device. In certain embodiments the device or devices are covered in a single casing. In certain embodiments a computer is connected to the single device or each of the devices, INAS 100 and the Detector Unit 101 respectively. The computer may be included in the device or may be operably coupled to the device or devices by an indirect link, such as a cable. The computer may monitor the irradiation amount and provide information and analysis about the amount of trace element found in the tissue.

The following are materials, methods, and examples of one embodiment of this technology. The following is not meant to be restrictive or limiting in any way.

Monte Carlo simulation. In one example, the system may use the Monte Carlo N-Particle (MCNP) code, which was developed by Los Alamos National Laboratory (LANL). MCNP code uses the Monte Carlo method to simulate the propagation of particles, including neutrons and photons. At thermal neutron energies, the binding of the scattering nucleus in a solid, liquid, or gas affects the cross-section and the angular and energy distributions of the scattered neutrons. When available, the S(α, β) data were included to better simulate thermal neutron interaction. The system may use MCNP5 version 1.6 of the code, and all input files may be checked with the VISED X_24E visual editor for geometric consistency or an equivalent. All the results discussed below have uncertainties of less than 5%.

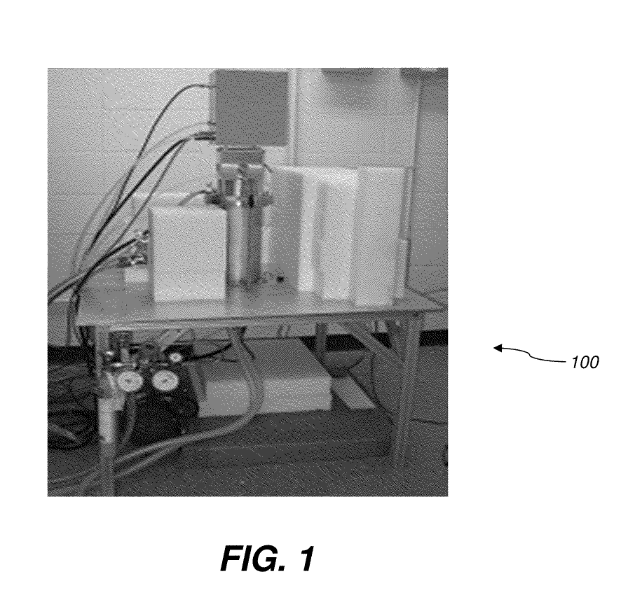

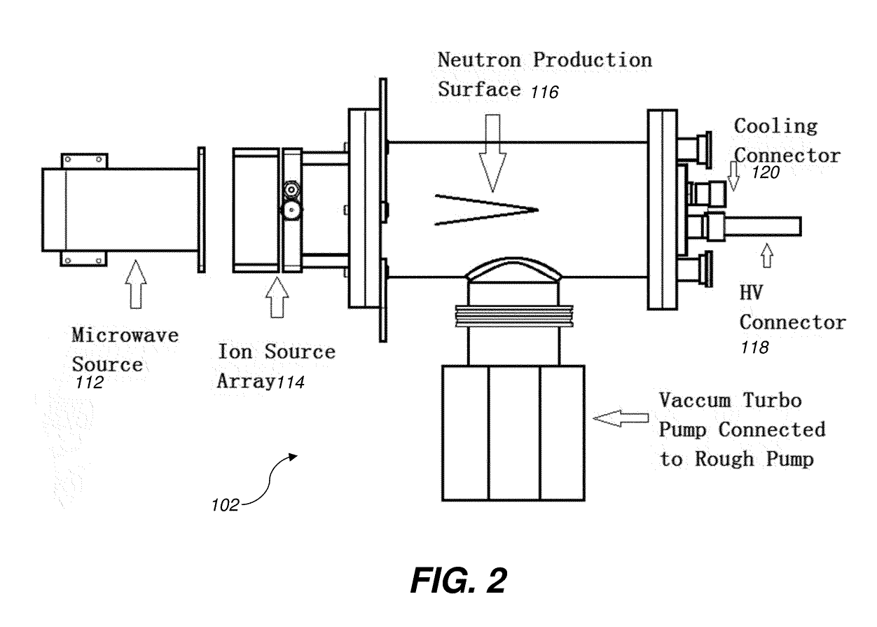

A DD neutron generator and neutron flux may be used. In one example, the neutron generator is a customized DD-109 manufactured by Adelphi Technology Inc. (Redwood, Calif.), but an equivalent is also acceptable. The main components of a DD neutron generator, such as generator 102, are a microwave source 112, an ion extractor 114, a neutron production surface 116, also referred to as a beam target, a power supply 118, and a heat exchanger cooling connector 120. Referring now to FIG. 1, which shows the DD neutron generator 102 installed in a lab, while FIG. 2 is a schematic plot of the generator head. A deuterium (D2) gas bottle was mounted beside the table to provide a continuous supply of deuterium gas when the system was operating. The gas line was vacuumed by a roughing pump and a turbo pump Like all DD neutron generators, the DD-109 employed in this example used the DD fusion reaction (²D+²D→⁴He→³He+n) and was driven by an ion beam supplied by a radio frequency-driven ion source, although an equivalent ion source is also acceptable.

The target for this example is made from titanium-coated copper and V-shaped, but targets with other shape configurations such as a flat surface, are also acceptable. Optionally to maximize the neutron production and lifetime of the target, the temperature of the titanium surface may be maintained by active cooling using liquid fluorinert or an equivalent cooling system. The V-shaped target is also designed for efficient cooling, as shown in FIG. 2. To shield the bremsstrahlung x-rays generated by the electrons emitted back to the plasma source from the primary ion interaction at the titanium target, 3 mm thick lead is placed around the generator head. Other materials of varying thickness may be used to shield neutrons, for example polyethylene or borated polyethylene. In further optimizations, the target is located between 2 mm and 0 mm from the moderator.

Neutron flux of up to 3×10⁹ neutrons/second can be produced with the above described generator, depending on the acceleration voltage and the ion current. In one example, the voltage varies from about 80 kV to about 125 kV, while the current varies from about 10 mA to about 13 mA. The neutron ambient dose equivalent can be obtained using a neutron survey meter and taking into account the ICRP 74 neutron flux-to-dose equivalent rate conversion factors. The NSN3 dosimeter's mono-energetic and continuous energy response is within 50% difference from thermal to 15 MeV neutrons.

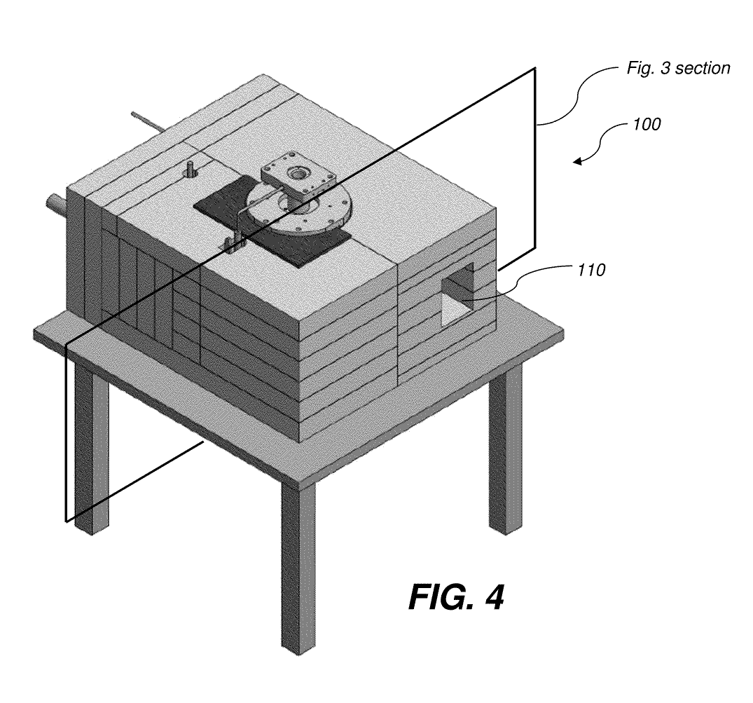

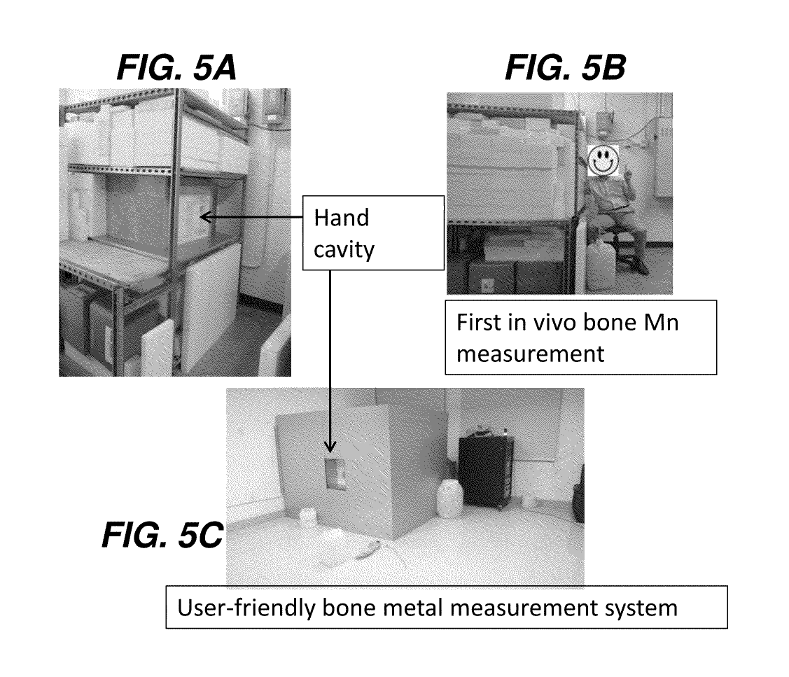

A Moderator, reflector, and shielding system setup of the DD neutron generator. Based on the MC simulation results presented previously, an optimized moderator, reflector, and shielding system was built including a cavity for the irradiation of human tissue. In certain embodiments this cavity may be used to measure trace elements in a human hand. One configuration comprises of 5 cm of paraffin as the moderator and greater than 10 cm of paraffin as the reflector. In other embodiments the configuration may comprise between 3 and 7 cm of paraffin as the moderator, and between 10 and 25 cm for the reflector. In the system used in the present example, polyethylene is used instead of paraffin because paraffin is flammable. In another embodiment, 5 cm of polyethylene may be used as the moderator; 10 cm of polyethylene was used as the reflector; and the shielding structure may be made of greater than 30 cm of polyethylene. The neutron dose outside of the shielding was measured to be 2-5 mR/hour based on this configuration. In other embodiments the moderator may comprise between 2 and 7 cm of polyethylene, the reflector may comprise between 6 and 14 cm of polyethylene, and the shielding may comprise between 28 and 40 cm of polyethylene. FIG. 4 shows a setup of the DD neutron generator with the polyethylene moderator, reflector, and shielding system, with part of the shielding removed to present a better view of the cavity.

In certain embodiments, the cavity is between about 6 inches to about 20 inches in length. In some aspects the cavity is between about 4 inches and 10 inches in width. In other embodiments, the cavity may be round-like in shape. In some aspects the cavity comprises a cavity shielding which comprises a material to shield surrounding tissues. In some embodiments the cavity shielding may be a bag which can be filled with water to insulate surrounding tissues, such as an arm, limiting exposure of dosing to the hand. The water bag cavity shielding may be adjusted to the subject's size and weight to ensure that the surrounding tissue was properly shielded, while not constricting or causing any discomfort for the subject.

In other embodiments a more compact shielding structure with a tighter fit around the generator head except on the side of the cavity may be used, where the gaps between the moderator blocks and reflector blocks change. In addition, graphite may be used for a reflector. In one embodiment the shielding 108 may be configured to fit together like LEGOS (interlocking blocks) where there are no gaps and the individual pieces snap together to provide stability. In other embodiments the shielding is a solid single piece. In other embodiments, the shielding 108 is a single piece or a plurality of pieces, which are then covered by another smooth piece to act as a casing around the entire system.

Manganese-doped human hand phantoms. Five Mn-doped hand phantoms were manufactured and used in testing scenarios according to the present disclosure. The Mn concentrations in the phantoms were 0, 5, 10, 15, and 20 μg Mn/g bone (which corresponds to 0, 22, 44, 66, and 88 μg Mn/g Ca). Other elements in bone that might interfere with the spectrum through neutron activation were also added to the phantoms to better simulate real human hands. The concentration of each element in the bones of the hand was calculated based on ICRP publication 23's gross and element content of the cortical bone of a reference human male. Table 1 lists all the elements included and the weight of their chemical compounds.

TABLE 1
Mass of each element and compound used in the hand phantoms.

| | Ca | Cl | Na | Mg | Mn |
|---|---|---|---|---|---|
| Reaction | ⁴⁸Ca(n, | ³⁷Cl(n, | ²³Na(n, | ²⁶Mg(n, | ⁵⁵Mn(n, |
| | γ)⁴⁹Ca | γ)³⁸Cl | γ)²⁴Na | γ)²⁷Mg | γ)⁵⁶Mn |
| Mass | 13.925 | 1.205 | 1.29 | | 0, 5, 10, 15, |
| | | | | | 20, ppm |
| Compound added | CaSO₄ | NH₄Cl | NaNO₃ | MgSO₄ | Mn(NO₃)₂ |
| Mass | 50.4 g | 1.82 g | 4.77 g | 1.2 g | 0, 1.1, 2.2, |
| | | | | | 3.3, 4.5 mg |

All the chemical compounds were first diluted in distilled water before being added to the matrix to ensure a better homogeneity of the elements in the phantoms. The phantoms were then dried in the hood for one day. These phantoms were bone-equivalent phantoms.

In vivo neutron activation analysis. With the system shown in FIG. 4, Mn concentrations present in the human hand phantoms can be noninvasively determined using in vivo neutron activation analysis (IVNAA). During neutron activation, characteristic γ-rays are produced following the radioactive decay of the product from an ^{A}X(n,γ)^{(A+1)}X nuclear reaction. By collecting the characteristic Trays and calculating their total counts, the concentration of the element of interest can be determined. For Mn quantification, neutrons interact with ⁵⁵Mn and produce ⁵⁶Mn with a thermal neutron capture cross section of 13.3 barns. Unstable ⁵⁶Mn atoms decay to ⁵⁶Fe, which emits 847 keV characteristic γ-rays. These γ-rays can then be collected by a γ-ray detection system. ⁵⁶Mn's relatively long half-life of 2.58 hours allows for delayed γ counting. Other elements may be analyzed in the same way, however, it will be recognized that the time between dosing and analyzing may be different and that different signatures will be detected depending on the trace element. One of ordinary skill in the art will understand the decay rates and the decay signal associated with each trace element, and will be able to use the device to produce a protocol to fit his or her specific needs.

Using the fm4 card in MCNP5 or an equivalent, the probability of the activated nucleus can be obtained. Together with the activation equation, the simulated total γ-ray counts can be expressed as:

C_{Total}=N×γε×S×D×C (1)

where C_{Total} is the γ-ray counts that will be measured by the γ-ray detector; N is the total activated ⁵⁶Mn number from the simulation result; γ is the branch ratio of the γ-rays; c is the absolute detection efficiency; S(=1−e^{-λt}^{ᵢ}) is the saturation factor with tᵢ representing irradiation time; D(=e^{-λt}^{_{d}}) is the decay factor with t_{d} representing decay time; and C(=1−e^{-λt}^{_{c}})/λ is the counting factor with t_{c} representing counting time. C_{Total} can be compared to experiment results to test the consistency of the simulation and experimental results.

The irradiation, decay, and counting time can also be optimized to determine the best time sequence. In this example, 10 minutes of irradiation time was used to allow for an acceptable dose to the hand; 5 minutes of decay time to collect a spectrum for calcium (Ca); and 30 minutes of measurement time in consideration of the time that a human subject could be expected to sit relatively still to take the measurement. In other embodiments the subject may be irradiated for about 2 minutes to about 15 minutes, about 5 minutes to about 12 minutes, about 8 minutes to about 10 minutes. In certain aspects the decay time may range from about 30 seconds to about 10 minutes, from about 1 minute to about 9 minutes, from about 2 minutes to about 8 minutes, from 4 minutes to about 6 minutes, or greater than 5 minutes. In certain aspects, the measurement time may be from about 5 minutes to about 45 minutes, from about 10 minutes to about 40 minutes, from about 15 minutes to about 30 minutes, from about 20 minutes to about 25 minutes, or up to one hour. The variation will depend on the tissue, subject, radiation source, trace element, and decay signal involved in the analysis, and one of ordinary skill in the art will recognize a need to adjust parameters to get an accurate dosing and measurement.

A sample of pure gold (Au) foil and an Mn-doped hand phantom were irradiated by the DD-based neutron generator system and then measured by the Detector Unit. In one embodiment the Detector Unit is an HPGe γ-ray detection system or an equivalent. The same scenarios were also simulated using the MC simulation model. The results from the MC simulations and the experiments were then compared. This is to validate the results from MC simulations.

HPGe detector and gamma spectrum analysis in the Detector Unit. A high-efficiency HPGe detector may be used in this technology for γ-ray detection. It is a model GMX90P4-ST HPGe detector with a relative efficiency of 100%, but other equivalents may be acceptable. The detector may be cooled by an electromechanical cooler (Ortec, Oak Ridge, Tenn.), or other coolers that are known or may be used to cool HPGe detectors is acceptable. Lead bricks were mounted around the detector to reduce the background signal, but other materials such as polyethylene, borated polyethylene, or a combination thereof may be used to reduce back ground signal. The material ranges in a thickness of about 5 cm to about 15 cm. The DSPEC PLUS digital box was used for signal processing, and Maestro γ-ray spectroscopy software was used for signal collection, but equivalents may also be used. The efficiency of the system was calibrated using a multi-radionuclide calibration source with known activities. The efficiency equation was obtained as:

efficiency=1.0548×energy(keV)^{-0.688} (2)

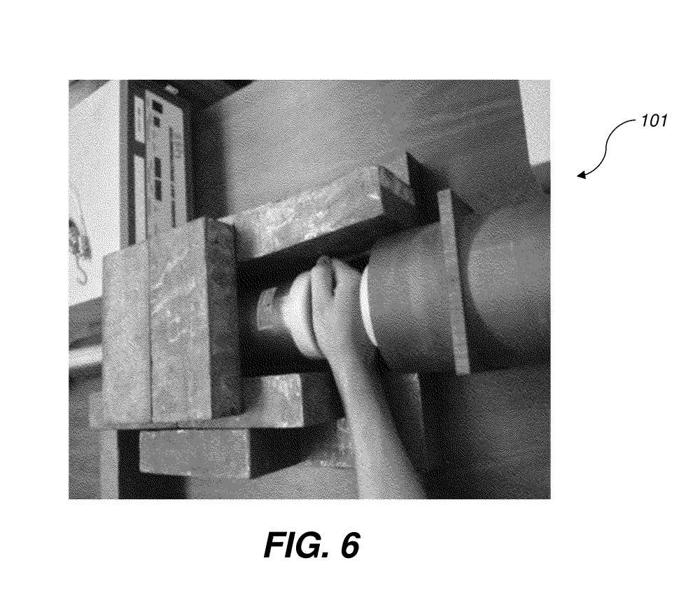

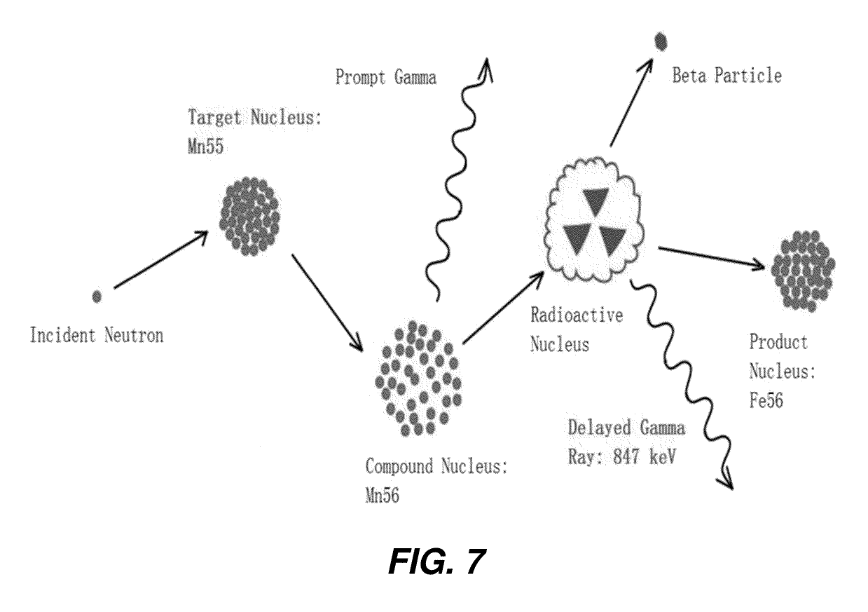

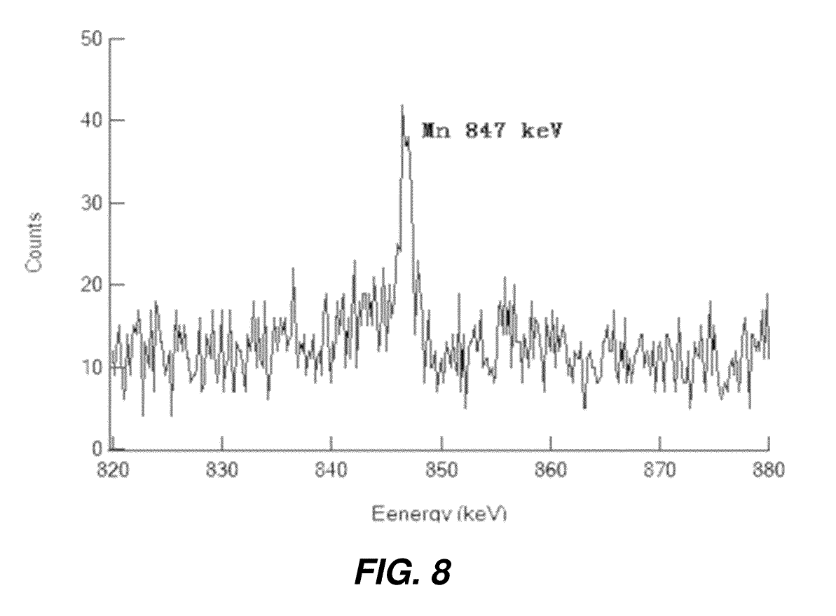

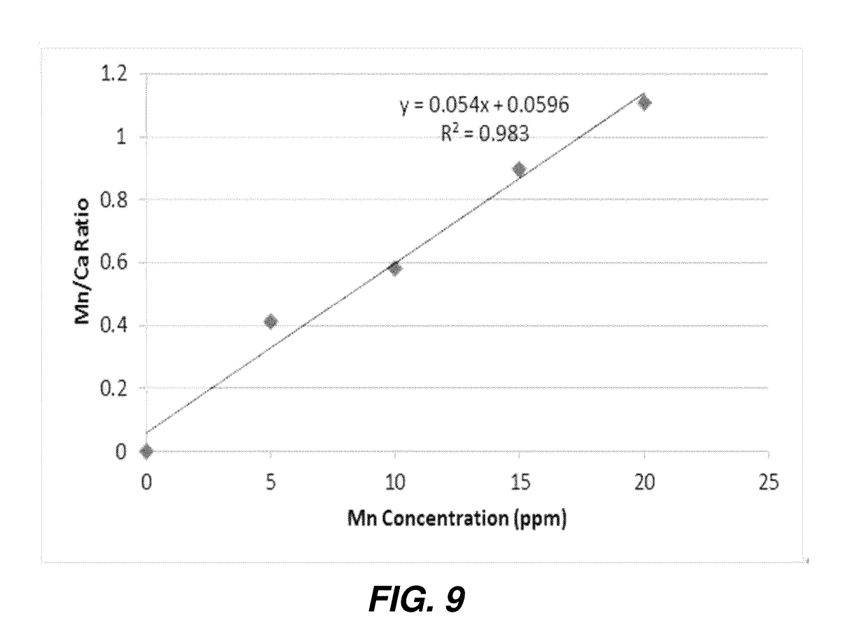

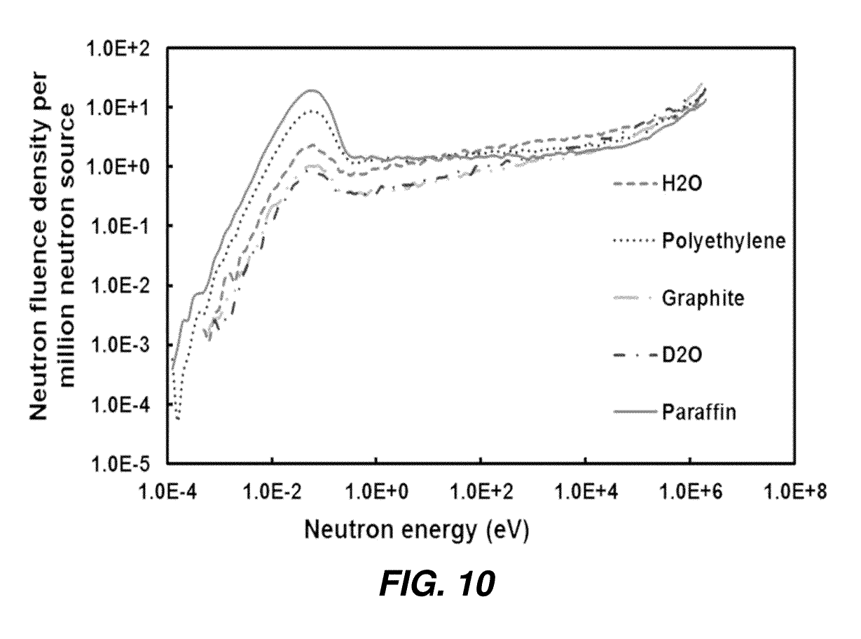

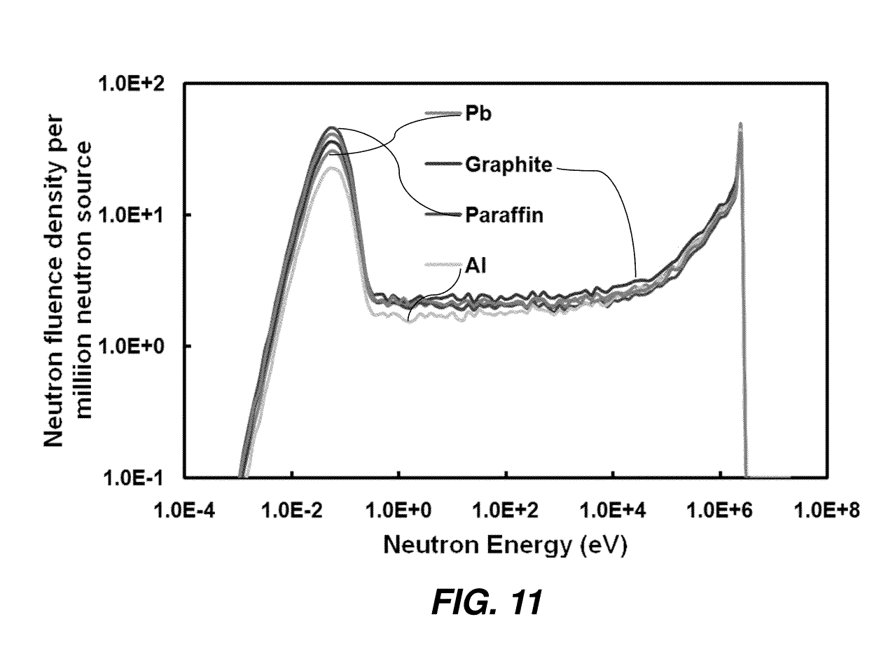

at 5 cm away from the detector's window. FIG. 6 shows two high-efficiency detectors with a possible configuration of how a hand could be measured. In certain embodiments, up to six detectors may be used to increase the sensitivity of the decay signal capture by the Detector Unit. In some embodiments the Detector unit may have between one and 6 detectors, 1 and 5 detectors, 1 and 4 detectors, 1 and 3 detectors, 1 and 2 detectors, 2 and 3 detectors, 2 and 4 detectors, 2 and 5 detectors, or 2 and 6 detectors. The detectors may be of any brand or make as long as they have an ability to measure the decay signal of the trace element of interest.

Gamma-ray spectrum analysis was performed using an in-house fitting procedure programmed in the commercial software package IGOR Pro 6 (Wave Metrics, Inc., Lake Oswego, Oreg.), but an equivalent may be used. The γ-ray peaks were fitted using the Gaussian function for net counts and an exponential function to account for background.

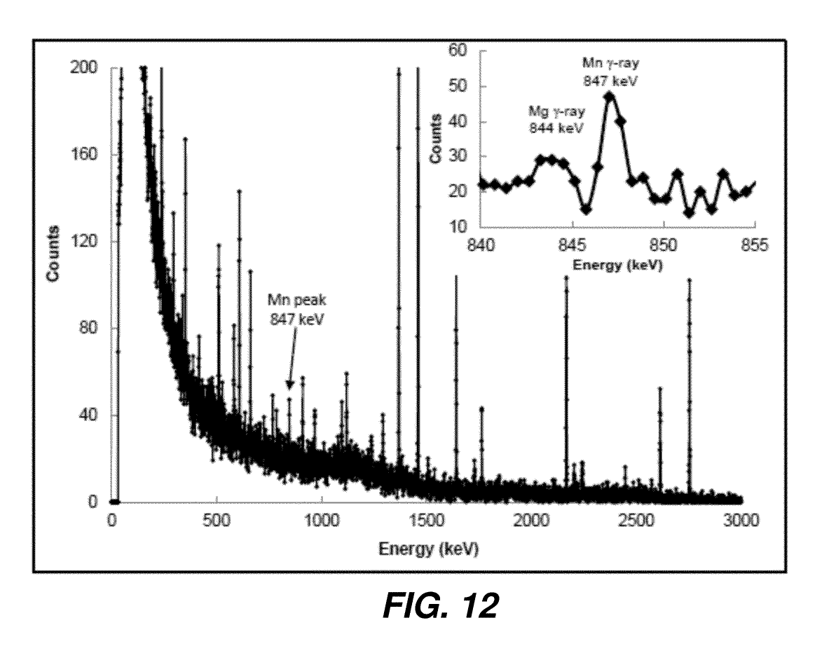

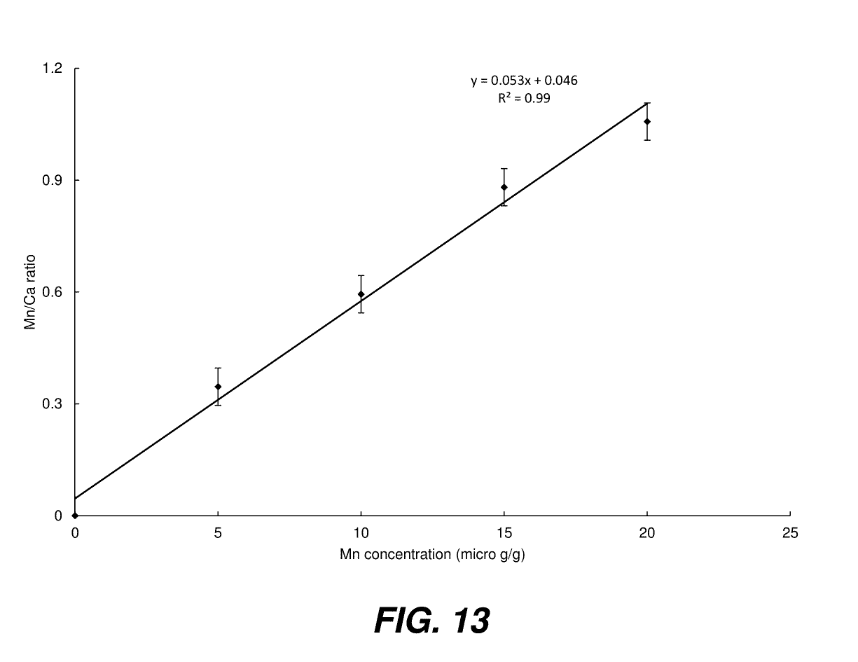

System calibration and the Mn/Ca ratio. The most straightforward way to calibrate the system for Mn quantification is to build a calibration line of ⁵⁶Mn γ-ray counts versus Mn concentration. However, this count would be affected by the thermalization of the neutrons within the samples, the thickness of the soft tissue in the subject (for example a hand), the weight of the tissue, and the slightly different irradiation geometries. Referring to FIGS. 12 and 13, to account for these differences in this specific instance of looking at bone, Mn γ-ray counts can be normalized to Ca γ-ray counts, since the concentration of Ca is relatively constant in bone. Thus, a calibration line representing the Mn/Ca ratio versus Mn concentration was established.

The above system and method may be implemented using a computer processor operatively connected to a memory and other computing components, and received or presented to a user using input/output devices such as an electronic display, keyboard and mouse. The processor can implement processes of various aspects described herein. The processor can be or include one or more device(s) for automatically operating on data, e.g., a central processing unit (CPU), microcontroller (MCU), desktop computer, laptop computer, mainframe computer, personal digital assistant, digital camera, cellular phone, smartphone, or any other device for processing data, managing data, or handling data, whether implemented with electrical, magnetic, optical, biological components, or otherwise. The processor can include Harvard-architecture components, modified-Harvard-architecture components, or Von-Neumann-architecture components.

The phrase “communicatively connected” includes any type of connection, wired or wireless, for communicating data between devices or processors. These devices or processors can be located in physical proximity or not.

Various aspects described herein may be embodied as systems or methods. Accordingly, various aspects herein may take the form of an entirely hardware aspect, an entirely software aspect (including firmware, resident software, micro-code, etc.), or an aspect combining software and hardware aspects These aspects can all generally be referred to herein as a “service,” “circuit,” “circuitry,” “module,” or “system.” Furthermore, various aspects herein may be embodied as computer program products including computer readable program code stored on a tangible non-transitory computer readable medium. Such a medium can be manufactured as is conventional for such articles, e.g., by pressing a CD-ROM. The program code includes computer program instructions that can be loaded into a computer processor to cause functions, acts, or operational steps of various aspects herein to be performed by the processor. Computer program code for carrying out operations for various aspects described herein may be written in any combination of one or more programming language(s).

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

What is claimed is:

1. An in vivo neutron activation analysis (NAA) system comprising:
a neutron generator;
a moderator operably coupled to the neutron generator;
a cavity;
a reflector operably coupled to the neutron generator, moderator, and cavity; and
a shielding operably coupled to the neutron generator, moderator, cavity, and reflector.

2. The system of claim 1, wherein the neutron generator is positioned vertically.

3. The system of claim 1, wherein the shielding includes components comprising high density polyethylene (HDPE), borated HDPE, and lead.

4. The system of claim 1, wherein the cavity is round.

5. The system of claim 1, wherein the cavity includes a cavity shielding.

6. The system of claim 1, wherein the shielding is configured to lock or snap together.

7. The system of claim 1, wherein the neutron generator generates a range of neutrons between about 1×10⁸ to 1×10¹¹ neutrons per second.

8. The system of claim 1, wherein the moderator includes components made of polyethylene (PE).

9. The system of claim 1, wherein the reflector includes components made of graphite or polyethylene (PE).

10. A method of detecting trace elements in bone comprising:
modifying 2.45 MeV neutrons with a INAS system;
irradiating the bone and trace elements included in the bone with neutrons;
generating neutron capture induced gamma-ray signals from the trace elements;
detecting the signals from the trace elements using a gamma-ray detection system; and
analyzing the signals to determine the amount of the trace elements in the bone.

11. The method of claim 10, wherein the bone is the bone of an animal.

12. The method of claim 10, wherein the neutrons are applied to a live animal.

13. The method of claim 10, wherein the neutrons are generated in a range between about 1×10⁸ to about 1×10¹¹.

14. The method of claim 10, wherein detecting the gamma-ray signal uses at least one detector element.

15. The method of claim 10, wherein the trace elements are aluminum, magnesium, iron, manganese, titanium, fluorine, or gadolinium.

16. The method of claim 10, wherein the animal bone is from a human.

17. The method of claim 16, wherein the animal bone is from a human hand.
